(19) United States
(12) Patent Application Publication
(10) Pub. No.: US 2010/0094186 A1
(43) Pub. Date: Apr. 15, 2010

(54) Orthotic boot

(76) Inventor: Terence Brown, Milwaukee, WI (US)

Correspondence Address: Terence Brown, 7450 W. Glenbrook Rd., Milwaukee, WI, 53223, US

(21) Appl. No.: 12/587,409

(22) Filed: Oct. 6, 2009

Related U.S. Application Data
(60) Provisional application No. 61/103,112, filed on Oct. 6, 2008.

(51) Int. Cl. A61F 5/01

(52) U.S. Cl. 602/16

(57) ABSTRACT

An orthotic boot comprised of highly durable polymer material which is light weight and very durable for limb protection. There are locks positioned on the medial and lateral sides of the foot portion and the shin portion to secure the device to the limb; locks are made out of stainless steel to prevent rusting. There is a breathable inner foam lining within the device that provides comfort and allows the skin to breathe. There is a durable stainless steel hinge positioned at the tip of the foot portion of the device that allows the device to open and close. The sole is constructed out of non-skid material for stability and safety. There is a energy return system built into the sole for maximum propulsion during walking; a gel containing air pockets is compressed during walking and returns to its normal shape when the foot is lifted releasing the stored energy.

### BACKGROUND OF THE INVENTION

1. Field of the Invention

The present invention relates to an orthotic boot, and more particularly, relates to an orthotic boot having a front hinge for easy access and removal.

2. Discussion of the Related Art

Orthotic boots and braces are used for individuals who require the help of orthotics due to neurological or muscular disorders that affect the lower extremities, or due to accident or injury. There are numerous orthotic boots available, most of which comprise a footbed portion having splint portions on either side, and some closure system, typically composed of Velcro straps. However, traditional orthotic boots and braces are difficult to get into and remove, making wear of the devices more challenging.

### SUMMARY OF THE INVENTION

The orthotic device of the present invention overcomes these problems by providing a boot having a foot support portion and a restraint portion that are hingedly connected at the toe portion of each. The device of the present invention preferably also provides an energy return system enabling the wearer to walk more comfortably while wearing the device.

### BRIEF DESCRIPTION OF THE DRAWINGS

Preferred exemplary embodiments of the invention are illustrated in the accompanying drawings, in which like reference numerals represent like parts throughout, and in which:

FIG. 1 is a front view of a preferred embodiment of the boot of the invention;

FIG. 2 is a side view of the boot shown in FIG. 1; and

FIG. 3 is a side view of the boot of FIG. 1 shown in an open position with the front, restraint portion of the device in cut away for viewing the structure of the lining.

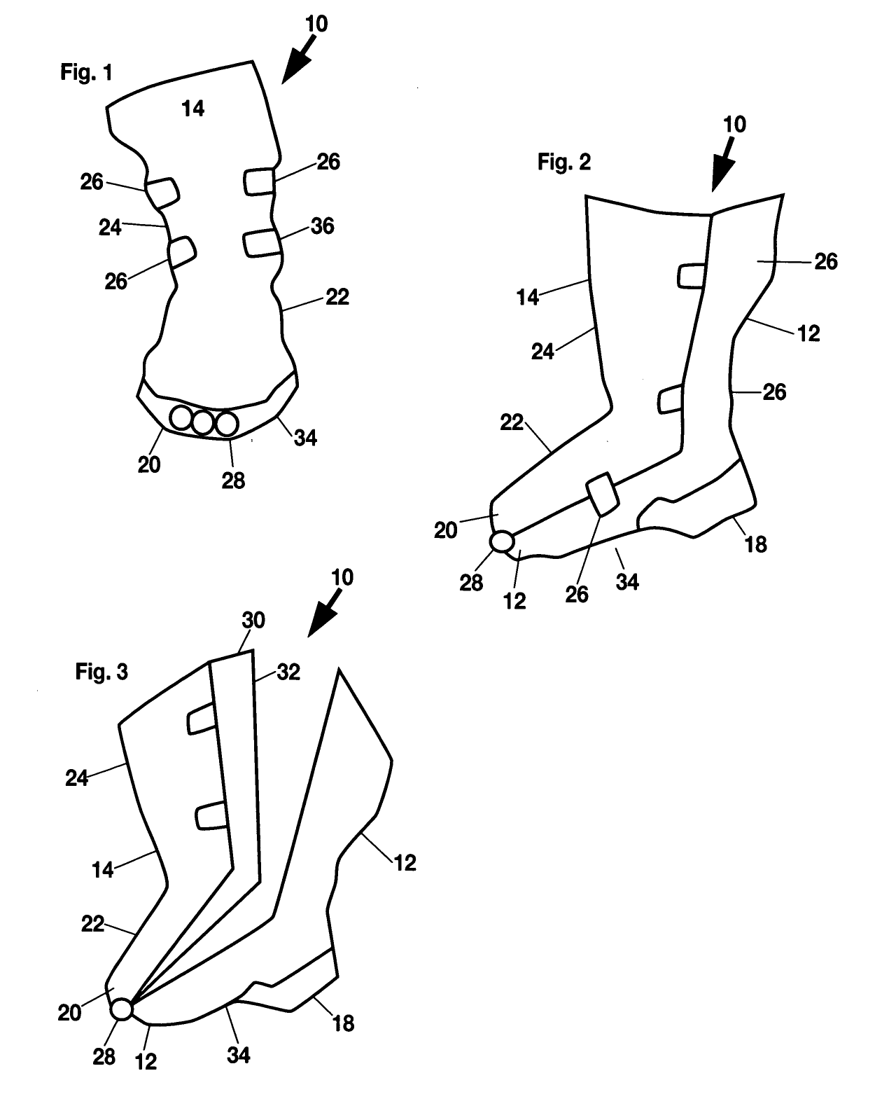

### DETAILED DESCRIPTION OF THE PREFERRED EMBODIMENTS

In a preferred embodiment of the present invention, a boot-shaped orthotic device 10 has a foot support portion 12 generally comprising the rear portion of device 10 and a restraint portion 14 generally comprising the front portion of device 10. Foot support portion 12 has a toe portion 16 and a heel portion 18.

Restraint portion 14 has a toe portion 20, top-of-foot portion 22, and a shin portion 24. Clamping locks 26 are provided to lock support portion 12 to restraint portion 14. Locks 26 are preferably provided on restraint portion 14 and are constructed of metal or polymer or any suitably strong and rigid material.

A hinge 28 connects foot support portion 12 and restraint portion 14 at the respective toe portions 16, 20 of each. Hinge 28, like locks 26, is constructed of metal or polymer or any suitably strong and rigid material.

Orthotic device 10 is constructed of a highly durable plastic or polymer material and has a breathable inner foam lining 30, preferably encasing a breathable cushion 32, which provides comfort and breathability. Device 10 is preferably provided with air holes (not shown) for circulation and ventilation. Sole 34 is constructed of non-skid material for stability and safety.

Orthotic device 10 preferably also features an energy return system comprising a gel pad (not shown) within or adjacent sole 34. The energy return system allows the wearer of device 10 to walk more easily in the device by taking advantage of the wearer's natural gait. Specifically, the gel pad absorbs the energy from the walker's foot strike—the loading portion of the gait cycle—and returns that energy to the walker for extra boost in the pre-swing portion of the gait cycle. The extra propulsion of the energy return system is particularly advantageous for wearers of an orthotic device since the devices are often heavy and/or bulky and require additional leg stress to lift while walking.

The embodiment described herein explains the best known mode of practicing the invention and will enable others skilled in the art to utilize the invention, but should not be considered limiting. Rather, it should be understood that the invention is not limited to the details of construction and arrangements of the components set forth herein, but is capable of other embodiments and of being practiced or carried out in various ways, and all such modifications and variations are within the scope of the claims set forth below. Further, various elements or features discussed or shown herein may be combined in ways other than those specifically mentioned, and all such combinations are likewise within the scope of the invention.

I claim:

1. An orthotic device comprising:
a foot support portion having a toe portion, a heel portion, and a sole portion;
a restraint portion having a toe portion, a top-of-foot portion, and a shin portion; and
a hinge connecting the toe portion of the foot support portion and the toe portion of the restraint portion.

2. A device as in claim 1, further comprising an energy return system adjacent the sole portion.
